(19) United States
(12) Patent Application Publication
(10) Pub. No.: US 2026/0106037 A1
(43) Pub. Date: Apr. 16, 2026

(54) INCORPORATING CLINICAL RISK INTO BIOMARKER-BASED ASSESSMENT FOR CANCER PRE-SCREENING

(71) Applicant: Delfi Diagnostics, Inc., Baltimore, MD (US)

(72) Inventors: Peter B. Bach, Baltimore, MD (US); Debbie M. Jakubowski, Baltimore, MD (US); Carter Portwood, Baltimore, MD (US); Niti Trivedi, Baltimore, MD (US)

(21) Appl. No.: 19/115,216

(22) PCT Filed: Oct. 6, 2023
(86) PCT No.: PCT/US2023/034705

§ 371 Date: Mar. 25, 2025

Related U.S. Application Data
(60) Provisional application No. 63/414,370, filed on Oct. 7, 2022.

(51) Int. Cl. G16H 50/30; C12N 15/10; C12Q 1/6806; C12Q 1/6869; C12Q 1/6886; G16B 30/00; G16H 20/10; G16H 20/40

(52) U.S. Cl. CPC G16H 50/30; C12N 15/1093; C12Q 1/6806; C12Q 1/6869; C12Q 1/6886; G16B 30/00; G16H 20/10; G16H 20/40; C12Q 2600/106

(57) ABSTRACT

Methods for pre-screening subjects for cancer are FIG. 1 disclosed. Genetic risks associated with genetic signatures are determined by sequencing and analyzing cell free DNA (“cfDNA”) fragments present in the subject's blood sample. Clinical risk is determined based on factors such as age, sex and race. In some cases, clinical factors specific to certain cancers such as smoking status are incorporated. Improved lung cancer pre-screening results are provided by incorporating clinical risk into Excluded Randomized to the genomic risk analysis, enabling the same number of positive x-ray study arm cancer detections using a lower number of LDCT lung cancer screens.

### CROSS REFERENCE TO RELATED APPLICATIONS

This application claims the benefit of priority under 35 U.S.C. § 119(e) of U.S. Provisional Patent Application Ser. No. 63/414,370 filed on Oct. 7, 2022. The disclosure of the prior application is considered part of and is herein incorporated by reference in the disclosure of this application in its entirety.

### BACKGROUND OF THE INVENTION

### Field of the Invention

The invention relates generally to cancer pre-screening and more specifically to the improvement of cancer pre-screening results by incorporating clinical risk factors into the analysis of cell free DNA (“cfDNA”).

### Background Information

Blood-based biomarker assessments that identify genomic signatures of cancer have the potential to improve the early detection of cancer. In particular, cancer pre-screening using blood samples in which cfDNA fragments are sequenced and aligned to the genome can provide information such as the composition of the cfDNA population, the genomic location of the cfDNA fragments, physical characteristics such as fragment size and fragment ends, as well as the presence of changes indicative of cancer such as copy number changes, microsatellite instabilities or other known cancer-causing genetic variations.

### SUMMARY OF THE INVENTION

The present invention is based on the seminal discovery that incorporating individual-level clinical risk with genomic signatures of cancer improves the identification of subjects who are most likely to have cancer found by screening. Among other things, the present disclosure demonstrates that the incorporation of clinical risk factors for lung cancer into an analysis of cfDNA testing improved the identification of subjects who are most likely to have positive confirmation of lung cancer by standard low dose computed tomography (“LDCT”) lung cancer screening.

In clinical use, genomic signatures of cancer are typically interpreted using a cutoff point, above which results are positive, and below which they are negative. However, relying solely on a genomic signature ignores underlying clinical risk factors associated with the subject. The present disclosure describes methods of blood sample-based cancer pre-screening. Individual-level clinical risk is matched with genomic signatures of cancer, thereby improving the identification of subjects who are most likely to have cancer found by standard cancer screening methods.

In one embodiment, the present invention provides a method of predicting the cancer status of a subject which includes determining a clinical risk score for the subject; determining a genomic risk score for the subject; and combining the clinical risk score with the genomic risk score, thereby predicting the cancer status of the subject. In one aspect, the present invention provides a method wherein the clinical score includes the age, sex and/or race of the subject. In a further aspect, the genomic risk score includes cell free DNA (cfDNA) fragment size density data from the subject. In certain aspects, the cfDNA is obtained from a blood sample from the subject.

In certain aspects, determining the cfDNA fragment size density data for the subject includes: processing a sample from the subject including cfDNA fragments into libraries; subjecting the libraries to low-coverage whole genome sequencing to obtain sequenced fragments; mapping the sequenced fragments to a genome to obtain windows of mapped sequences; analyzing the windows of mapped sequences to determine cfDNA fragment lengths; and generating the cfDNA fragment size density data.

In certain aspects, the cfDNA fragment size density data is calculated for one or more subgenomic interval(s). In additional embodiments, a cfDNA fragmentation profile is determined for each subgenomic interval. In further aspects, the cfDNA fragment size density data includes a curve. In some such aspects, the cfDNA fragment size density curve from the subject is compared to a cfDNA fragment size density curve from a known healthy subject and/or a known cancer patient. In more aspects, the cfDNA fragmentation profile includes a fragment size of greatest frequency. In further aspects, the cfDNA fragmentation profile includes a fragment size distribution having fragment sizes of varying frequency. In some aspects, the cfDNA fragmentation profile includes the sequence coverage of small cfDNA fragments in windows across the genome. In further aspects, the cfDNA fragmentation profile includes the sequence coverage of large cfDNA fragments in windows across the genome. In other aspects, the cfDNA fragmentation profile includes the sequence coverage of small and large cfDNA fragments in windows across the genome. In certain aspects, the mapped cfDNA fragment sequences include tens to thousands of genomic windows. In some such aspects, the windows are non-overlapping windows. In other aspects, the windows each include about 5 million base pairs. In further aspects, the cfDNA fragmentation profile covers the entire genome.

In another aspect, incorporating the clinical risk score and the genomic risk score results in a greater number of positive cancer diagnoses per subject screenings, as compared to using clinical risk score or genomic risk score alone. In certain aspects, the number of subject screenings needed to achieve one positive cancer diagnosis is reduced by at least an average of about 5%, 15%, 25%, 35%, 45%, 55%, 65%, 75% or more, as compared to using clinical risk score alone. In additional aspects, the number of subject screenings needed to achieve one positive cancer diagnosis is reduced by at least an average of about 5%, 10%, 15%, 20%, 25% or more as compared to using genetic risk score alone. In an additional aspect, incorporating the clinical risk score with the genomic risk score results in improved discrimination between subjects predicted to have a high risk for cancer and subjects precited to have a low risk for cancer. In another aspect, incorporating the clinical risk score with the genomic risk score results in a higher specificity of cancer prediction as compared to using clinical risk score alone or genomic risk score alone. In further aspects, the sensitivity of cancer prediction is at least about 50%, 60%, 70%, 80%, 90% or more.

In further aspects, the cancer is lung cancer. In some such aspects, the clinical risk score for the subject is determined from data including the age, sex, race, smoking status, number of pack years, and smoking duration of the subject. In certain aspects, the clinical risk score for the subject is determined from data including the Bach lung cancer incidence model as described in Bach, P. B., et al. J NATL CANCER INST. 95(6):470-8 (2003), which is herein incorporated with respect to its description of the Bach lung cancer incidence model. In an additional aspect, incorporating the clinical risk score with the genomic risk score results in a combined score that increases as the subject's risk for cancer increases. In certain aspects, the subject with an increased risk for cancer is administered a cancer treatment.

### BRIEF DESCRIPTION OF THE DRAWINGS

FIG. 1 illustrates a flow diagram of participant disposition.

FIG. 2 provides demographic and clinical characteristics of the participants. “IQR” stands for “interquartile range” and “n/a” stands for “not applicable.”

FIG. 3 illustrates binary clinical risk by cancer status.

FIG. 4 illustrates a distribution of clinical risk by cancer status. The line inside the rectangular box represents the median (the line) and IQR (the rectangular box), respectively.

FIG. 5 illustrates a distribution of simulated genomic risk by clinical risk status. The line inside the rectangular box represents the median (the line) and IQR (the rectangular box), respectively.

FIG. 6 illustrates the number of CT scans needed to detect one lung cancer by type of risk estimation.

FIG. 7 illustrates a model specificity (95% CI) to detect lung cancer, at 80% sensitivity.

FIG. 8 illustrates predicted probabilities of lung cancer diagnosis, using clinical risk.

FIG. 9 illustrates predicted probabilities of lung cancer diagnosis, using clinical and genomic risk.

FIG. 10 illustrates an example computer 800 that may be used in predicting the cancer status of a subject.

### DETAILED DESCRIPTION OF THE INVENTION

The present invention is based on the seminal discovery that incorporating individual-level clinical risk with a genomic signature improves the identification of subjects who are most likely to have cancer found by screening. Among other things, the present disclosure demonstrates that the incorporation of clinical risk factors for lung cancer into an analysis of cfDNA testing improved the identification of subjects who are most likely to have positive confirmation of lung cancer by low dose computed tomography (“LDCT”) lung cancer screening.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

In one embodiment, the present invention provides a method of predicting the cancer status of a subject which includes determining a clinical risk score for the subject; determining a genomic risk score for the subject; and combining the clinical risk score with the genomic risk score, thereby predicting the cancer status of the subject.

In one aspect, determining a clinical risk score comprises estimating a 1-year lung cancer risk for the subject. In one aspect, a 1-year lung cancer risk for the subject is determined using a Bach lung cancer incidence model. The Bach lung cancer incidence model is described in Bach, P. B., et al. J NATL CANCER INST. 95(6):470-8 (2003), which is herein incorporated with respect to its description of the Bach lung cancer incidence model. In one aspect, the clinical risk score is determined based on the subject's age, sex, asbestos exposure history, and smoking history. In one aspect, estimating a 1-year lung cancer risk for the subject comprises categorizing the subjects'cancer risk as low clinical risk or high clinical risk. In one aspect, the 25th percentile of clinical risk can be used to distinguish low from high clinical risk. In one aspect, determining a clinical risk score comprises interrogating the subject regarding their age, sex, smoking history, asbestos exposure, history of obstructive lung disease, brand of cigarette smoked, type of asbestos exposed to, findings on chest x-ray, and exposure to radon or secondhand smoke or any combination thereof; determining the subject's cancer risk based on responses provided by the subject using Bach lung cancer incidence model; and assigning a clinical risk score for the subject.

In one aspect, the present invention provides a method wherein the clinical score includes the age, sex and/or race of the subject.

In a further aspect, the genomic risk score includes cell free DNA (cfDNA) fragment size density data from the subject. In certain aspects, determining the cfDNA fragment size density data for the subject comprises: processing a sample from the subject including cfDNA fragments into libraries; subjecting the libraries to low-coverage whole genome sequencing to obtain sequenced fragments; mapping the sequenced fragments to a genome to obtain windows of mapped sequences; analyzing the windows of mapped sequences to determine cfDNA fragment lengths; and generating the cfDNA fragment size density data.

In a further aspect the genomic risk score is determined based on the subject's cfDNA fragmentation profile. In one aspect, the cfDNA fragmentation profile may be being determined by: obtaining and isolating cfDNA fragments from the subject, sequencing the cfDNA fragments to obtain sequenced fragments, mapping the sequenced fragments to a genome to obtain windows of mapped sequences, and analyzing the windows of mapped sequences to determine cfDNA fragment lengths and generate the cfDNA fragmentation profile.

In certain aspects, the cfDNA is obtained from a blood sample from the subject.

In some aspects, determining the cfDNA fragment size density data for the subject includes: processing a sample from the subject including cfDNA fragments into libraries; subjecting the libraries to low-coverage whole genome sequencing to obtain sequenced fragments; mapping the sequenced fragments to a genome to obtain windows of mapped sequences; analyzing the windows of mapped sequences to determine cfDNA fragment lengths; and generating the cfDNA fragment size density data.

In some aspects, a cfDNA fragmentation profile may be being determined by: obtaining and isolating cfDNA fragments from the subject, sequencing the cfDNA fragments to obtain sequenced fragments, mapping the sequenced fragments to a genome to obtain windows of mapped sequences, and analyzing the windows of mapped sequences to determine cfDNA fragment lengths and generate the cfDNA fragmentation profile.

The methodology of the present invention is based on low coverage whole genome sequencing and analysis of isolated cfDNA. In one aspect, the data used to develop the methodology of the invention is based on shallow whole genome sequence data (1-2× coverage).

In some aspects, mapped sequences are analyzed in non-overlapping windows covering the genome. Conceptually, windows may range in size from thousands to millions of bases, resulting in hundreds to thousands of windows in the genome. 5 Mb windows were used for evaluating cfDNA fragmentation patterns as these would provide over 20,000 reads per window even at a limited amount of 1-2× genome coverage. Within each window, the coverage and size distribution of cfDNA fragments was examined. In some aspects, the genome-wide pattern from an individual can be compared to reference populations to determine if the pattern is likely healthy or cancer-derived.

In certain aspects, the mapped sequences include tens to thousands of genomic windows, such as 10, 50, 100 to 1,000, 5,000, 10,000 or more windows. Such windows may be non-overlapping or overlapping and include about 1, 2, 3, 4, 5, 6, 7, 8, 9 or 10 million base pairs.

In various aspects, a cfDNA fragmentation profile is determined within each window. As such, the invention provides methods for determining a cfDNA fragmentation profile in a subject (e.g., in a sample obtained from a subject).

In some aspects, a cfDNA fragmentation profile can be used to identify changes (e.g., alterations) in cfDNA fragment lengths. An alteration can be a genome-wide alteration or an alteration in one or more targeted regions/loci. A target region can be any region containing one or more cancer-specific alterations. In some aspects, a cfDNA fragmentation profile can be used to identify (e.g., simultaneously identify) from about 10 alterations to about 500 alterations (e.g., from about 25 to about 500, from about 50 to about 500, from about 100 to about 500, from about 200 to about 500, from about 300 to about 500, from about 10 to about 400, from about 10 to about 300, from about 10 to about 200, from about 10 to about 100, from about 10 to about 50, from about 20 to about 400, from about 30 to about 300, from about 40 to about 200, from about 50 to about 100, from about 20 to about 100, from about 25 to about 75, from about 50 to about 250, or from about 100 to about 200, alterations).

In various aspects, a cfDNA fragmentation profile can include a cfDNA fragment size pattern. cfDNA fragments can be any appropriate size. For example, in some aspects, a cfDNA fragment can be from about 50 base pairs (bp) to about 400 bp in length. As described herein, a subject having cancer can have a cfDNA fragment size pattern that contains a shorter median cfDNA fragment size than the median cfDNA fragment size in a healthy subject. A healthy subject (e.g., a subject not having cancer) can have cfDNA fragment sizes having a median cfDNA fragment size from about 166.6 bp to about 167.2 bp (e.g., about 166.9 bp). In some aspects, a subject having cancer can have cfDNA fragment sizes that are, on average, about 1.28 bp to about 2.49 bp (e.g., about 1.88 bp) shorter than cfDNA fragment sizes in a healthy subject. For example, a subject having cancer can have cfDNA fragment sizes having a median cfDNA fragment size of about 164.11 bp to about 165.92 bp (e.g., about 165.02 bp).

In some aspects, a dinucleosomal cfDNA fragment can be from about 230 base pairs (bp) to about 450 bp in length. As described herein, a subject having cancer can have a dinucleosomal cfDNA fragment size pattern that contains a shorter median dinucleosomal cfDNA fragment size than the median dinucleosomal cfDNA fragment size in a healthy subject. In some aspects, on average, cancer-free subjects have longer cfDNA fragments in the dinucleosomal range (average size of 334.75 bp) whereas subjects with cancer have shorter dinucleosomal cfDNA fragments (average size of 329.6 bp). As such, a healthy subject (e.g., a subject not having cancer) can have dinucleosomal cfDNA fragment sizes having a median cfDNA fragment size of about 334.75 bp. In some aspects, a subject having cancer can have dinucleosomal cfDNA fragment sizes that are shorter than dinucleosomal cfDNA fragment sizes in a healthy subject. For example, a subject having cancer can have dinucleosomal cfDNA fragment sizes having a median cfDNA fragment size of about 329.6 bp.

A cfDNA fragmentation profile can include a cfDNA fragment size distribution. As described herein, a subject having cancer can have a cfDNA size distribution that is more variable than a cfDNA fragment size distribution in a healthy subject. In some aspects, a size distribution can be within a targeted region. A healthy subject (e.g., a subject not having cancer) can have a targeted region cfDNA fragment size distribution of about 1 or less than about 1. In some aspects, a subject having cancer can have a targeted region cfDNA fragment size distribution that is longer (e.g., 10, 15, 20, 25, 30, 35, 40, 45, 50 or more bp longer, or any number of base pairs between these numbers) than a targeted region cfDNA fragment size distribution in a healthy subject. In some aspects, a subject having cancer can have a targeted region cfDNA fragment size distribution that is shorter (e.g., 10, 15, 20, 25, 30, 35, 40, 45, 50 or more bp shorter, or any number of base pairs between these numbers) than a targeted region cfDNA fragment size distribution in a healthy subject. In some aspects, a subject having cancer can have a targeted region cfDNA fragment size distribution that is about 47 bp smaller to about 30 bp longer than a targeted region cfDNA fragment size distribution in a healthy subject. In some aspects, a subject having cancer can have a targeted region cfDNA fragment size distribution of, on average, a 10, 11, 12, 13, 14, 15, 15, 17, 18, 19, 20 or more bp difference in lengths of cfDNA fragments. For example, a subject having cancer can have a targeted region cfDNA fragment size distribution of, on average, about a 13 bp difference in lengths of cfDNA fragments. In some aspects, a size distribution can be a genome-wide size distribution.

A cfDNA fragmentation profile can include a ratio of small cfDNA fragments to large cfDNA fragments and a correlation of fragment ratios to reference fragment ratios. As used herein, with respect to ratios of small cfDNA fragments to large cfDNA fragments, a small cfDNA fragment can be from about 100 bp in length to about 150 bp in length. As used herein, with respect to ratios of small cfDNA fragments to large cfDNA fragments, a large cfDNA fragment can be from about 151 bp in length to 220 bp in length. As described herein, a subject having cancer can have a correlation of fragment ratios (e.g., a correlation of cfDNA fragment ratios to reference DNA fragment ratios such as DNA fragment ratios from one or more healthy subjects) that is lower (e.g., 2-fold lower, 3-fold lower, 4-fold lower, 5-fold lower, 6-fold lower, 7-fold lower, 8-fold lower, 9-fold lower, 10-fold lower, or more) than in a healthy subject. A healthy subject (e.g., a subject not having cancer) can have a correlation of fragment ratios (e.g., a correlation of cfDNA fragment ratios to reference DNA fragment ratios such as DNA fragment ratios from one or more healthy subjects) of about 1 (e.g., about 0.96). In some aspects, a subject having cancer can have a correlation of fragment ratios (e.g., a correlation of cfDNA fragment ratios to reference DNA fragment ratios such as DNA fragment ratios from one or more healthy subjects) that is, on average, about 0.19 to about 0.30 (e.g., about 0.25) lower than a correlation of fragment ratios (e.g., a correlation of cfDNA fragment ratios to reference DNA fragment ratios such as DNA fragment ratios from one or more healthy subjects) in a healthy subject.

In certain aspects, the cfDNA fragment size density data is calculated for one or more subgenomic interval(s). In additional embodiments, a cfDNA fragmentation profile is determined for each subgenomic interval.

In further aspects, the cfDNA fragment size density data includes a curve. In some such aspects, the cfDNA fragment size density curve from the subject is compared to a cfDNA fragment size density curve from a known healthy subject and/or a known cancer patient.

In more aspects, the cfDNA fragmentation profile includes a fragment size of greatest frequency. In further aspects, the cfDNA fragmentation profile includes a fragment size distribution having fragment sizes of varying frequency.

In some aspects, the cfDNA fragmentation profile includes the sequence coverage of small cfDNA fragments in windows across the genome. In further aspects, the cfDNA fragmentation profile includes the sequence coverage of large cfDNA fragments in windows across the genome. In other aspects, the cfDNA fragmentation profile includes the sequence coverage of small and large cfDNA fragments in windows across the genome. In certain aspects, the mapped cfDNA fragment sequences include tens to thousands of genomic windows. In some such aspects, the windows are non-overlapping windows. In other aspects, the windows each include about 5 million base pairs. In further aspects, the cfDNA fragmentation profile covers the entire genome.

Certain aspects further comprise preparing a cell free DNA (cfDNA) fragmentation profile to predict the a cancer status of a subject. In certain aspects, preparing a cell free DNA (cfDNA) fragmentation profile to predict the a cancer status of a subject may comprise: obtaining a sample from the subject; processing the sample to obtain a plasma fraction; extracting and purifying nucleosome protected cfDNA fragments from the plasma fraction; processing the cfDNA fragments obtained from the sample obtained from the subject into sequencing libraries; and subjecting the sequencing libraries to whole genome sequencing to obtain sequenced fragments, wherein genome coverage is about 9× to 0.1×.

In another aspect, incorporating the clinical risk score and the genomic risk score results in a greater number of positive cancer diagnoses per subject screenings, as compared to using clinical risk score or genomic risk score alone.

In certain aspects, the number of subject screenings needed to achieve one positive cancer diagnosis is reduced by at least an average of about 5%, 15%, 25%, 35%, 45%, 55%, 65%, 75% or more, as compared to using clinical risk scores alone.

In additional aspects, the number of subject screenings needed to achieve one positive cancer diagnosis is reduced by at least an average of about 5%, 10%, 15%, 20%, 25% or more as compared to using genetic risk score alone.

In an additional aspect, incorporating the clinical risk score with the genomic risk score results in improved discrimination between subjects predicted to have a high risk for cancer and subjects precited to have a low risk for cancer.

In another aspect, incorporating the clinical risk score with the genomic risk score results in a higher specificity of cancer prediction as compared to using clinical risk score alone or genomic risk score alone. In further aspects, the sensitivity of cancer prediction is at least about 50%, 60%, 70%, 80%, 90% or more.

In further aspects, the cancer is lung cancer. In some such aspects, the clinical risk score for the subject is determined from data including the age, sex, race, smoking status, number of pack years, and smoking duration of the subject.

In certain aspects, the clinical risk score for the subject is determined from data including the Bach lung cancer incidence model as described Bach, P. B., et al. J NATL CANCER INST. 95(6):470-8 (2003), which is herein incorporated with respect to its description of the Bach lung cancer incidence model.

In further aspects, the cancer can be any stage cancer. In some aspects, a cancer can be an early stage cancer. In some aspects, a cancer can be an asymptomatic cancer. In some aspects, a cancer can be a residual disease and/or a recurrence (e.g., after surgical resection and/or after cancer therapy). A cancer can be any type of cancer. Examples of types of cancers that can be assessed, monitored, and/or treated as described herein include, without limitation, lung, colorectal, prostate, breast, pancreas, bile duct, liver, CNS, stomach, esophagus, gastrointestinal stromal tumor (GIST), uterus and ovarian cancer. Additional types of cancers include, without limitation, myeloma, multiple myeloma, B-cell lymphoma, follicular lymphoma, lymphocytic leukemia, leukemia and myelogenous leukemia. In some aspects, the cancer is a solid tumor. In some aspects, the cancer is a sarcoma, carcinoma, or lymphoma. In some aspects, the cancer is lung, colorectal, prostate, breast, pancreas, bile duct, liver, CNS, stomach, esophagus, gastrointestinal stromal tumor (GIST), uterus or ovarian cancer. In some aspects, the cancer is a hematologic cancer. In some aspects, the cancer is myeloma, multiple myeloma, B-cell lymphoma, follicular lymphoma, lymphocytic leukemia, leukemia or myelogenous leukemia.

In an additional aspect, incorporating the clinical risk score with the genomic risk score results in a combined score that increases as the subject's risk for cancer increases.

In certain aspects, the subject with an increased risk for cancer is administered a cancer treatment.

A cancer treatment can be any appropriate cancer treatment. One or more cancer treatments described herein can be administered to a subject at any appropriate frequency (e.g., once or multiple times over a period of time ranging from days to weeks). Examples of cancer treatments include, without limitation, surgical intervention, adjuvant chemotherapy, neoadjuvant chemotherapy, radiation therapy, hormone therapy, cytotoxic therapy, immunotherapy, adoptive T cell therapy (e.g., chimeric antigen receptors and/or T cells having wild-type or modified T cell receptors), targeted therapy such as administration of kinase inhibitors (e.g., kinase inhibitors that target a particular genetic lesion, such as a translocation or mutation), (e.g., a kinase inhibitor, an antibody, a bispecific antibody), signal transduction inhibitors, bispecific antibodies or antibody fragments (e.g., BiTEs), monoclonal antibodies, immune checkpoint inhibitors, surgery (e.g., surgical resection), or any combination of the above. In some aspects, a cancer treatment can reduce the severity of the cancer, reduce a symptom of the cancer, and/or to reduce the number of cancer cells present within the subject.

In some aspects, a cancer treatment can be a chemotherapeutic agent. Non-limiting examples of chemotherapeutic agents include: amsacrine, azacitidine, axathioprine, bevacizumab (or an antigen-binding fragment thereof), bleomycin, busulfan, carboplatin, capecitabine, chlorambucil, cisplatin, cyclophosphamide, cytarabine, dacarbazine, daunorubicin, docetaxel, doxifluridine, doxorubicin, epirubicin, erlotinib hydrochlorides, etoposide, fiudarabine, floxuridine, fludarabine, fluorouracil, gemcitabine, hydroxyurea, idarubicin, ifosfamide, irinotecan, lomustine, mechlorethamine, melphalan, mercaptopurine, methotrxate, mitomycin, mitoxantrone, oxaliplatin, paclitaxel, pemetrexed, procarbazine, all-trans retinoic acid, streptozocin, tafluposide, temozolomide, teniposide, tioguanine, topotecan, uramustine, valrubicin, vinblastine, vincristine, vindesine, vinorelbine, and combinations thereof. Additional examples of anti-cancer therapies are known in the art; see, e.g., the guidelines for therapy from the American Society of Clinical Oncology (ASCO), European Society for Medical Oncology (ESMO), or National Comprehensive Cancer Network (NCCN).

In various aspects, DNA is present in a biological sample taken from a subject and used in the methodology of the invention. The biological sample can be virtually any type of biological sample that includes DNA. The biological sample is typically a fluid, such as whole blood or a portion thereof with circulating cfDNA. In embodiments, the sample includes DNA from a tumor or a liquid biopsy, such as, but not limited to amniotic fluid, aqueous humor, vitreous humor, blood, whole blood, fractionated blood, plasma, serum, breast milk, cerebrospinal fluid (CSF), cerumen (earwax), chyle, chime, endolymph, perilymph, feces, breath, gastric acid, gastric juice, lymph, mucus (including nasal drainage and phlegm), pericardial fluid, peritoneal fluid, pleural fluid, pus, rheum, saliva, exhaled breath condensates, sebum, semen, sputum, sweat, synovial fluid, tears, vomit, prostatic fluid, nipple aspirate fluid, lachrymal fluid, perspiration, cheek swabs, cell lysate, gastrointestinal fluid, biopsy tissue and urine or other biological fluid. In one aspect, the sample includes DNA from a circulating tumor cell.

As disclosed above, the biological sample can be a blood sample. The blood sample can be obtained using methods known in the art, such as finger prick or phlebotomy. Suitably, the blood sample is approximately 0.1 to 20 ml, or alternatively approximately 1 to 15 ml with the volume of blood being approximately 10 ml. Smaller amounts may also be used, as well as circulating free DNA in blood. Microsampling and sampling by needle biopsy, catheter, excretion or production of bodily fluids containing DNA are also potential biological sample sources.

The methods and systems of the disclosure utilize nucleic acid sequence information and can therefore include any method or sequencing device for performing nucleic acid sequencing including nucleic acid amplification, polymerase chain reaction (PCR), nanopore sequencing, 454 sequencing, insertion tagged sequencing. In some aspects, the methodology or systems of the disclosure utilize systems such as those provided by Illumina, Inc, (including but not limited to HiSeq™ X10, HiSeq™ 1000, HiSeq™ 2000, HiSeq™ 2500, Genome Analyzers™, MiSeq™, NextSeq, NovaSeq 6000 systems), Applied Biosystems Life Technologies (SOLID™ System, Ion PGM™ Sequencer, ion Proton™ Sequencer) or Genapsys or BGI MGI and other systems. Nucleic acid analysis can also be carried out by systems provided by Oxford Nanopore Technologies (GridiON™, MiniON™) or Pacific Biosciences (Pacbio™ RS II or Sequel I or II).

The present invention includes systems for performing steps of the disclosed methods and is described partly in terms of functional components and various processing steps. Such functional components and processing steps may be realized by any number of components, operations and techniques configured to perform the specified functions and achieve the various results. For example, the present invention may employ various biological samples, biomarkers, elements, materials, computers, data sources, storage systems and media, information gathering techniques and processes, data processing criteria, statistical analyses, regression analyses and the like, which may carry out a variety of functions.

Accordingly, the invention further provides a system for predicting the cancer status of a subject. In various aspects, the system includes: (a) a sequencer configured to generate a low-coverage whole genome sequencing data set for a sample; and (b) a computer system and/or processor with functionality to perform a method of the invention.

In some aspects, the computer system further includes one or more additional modules. For example, the system may include one or more of an extraction and/or isolation unit operable to select suitable genetic components analysis, e.g., cfDNA fragments of a particular size.

In some aspects, the computer system further includes a visual display device. The visual display device may be operable to display a curve fit line, a reference curve fit line, and/or a comparison of both.

Methods of predicting the cancer status of a subject according to various aspects of the present invention may be implemented in any suitable manner, for example using a computer program operating on the computer system. As discussed herein, an exemplary system, according to various aspects of the present invention, may be implemented in conjunction with a computer system, for example a conventional computer system comprising a processor and a random access memory, such as a remotely-accessible application server, network server, personal computer or workstation. The computer system also suitably includes additional memory devices or information storage systems, such as a mass storage system and a user interface, for example a conventional monitor, keyboard and tracking device. The computer system may, however, include any suitable computer system and associated equipment and may be configured in any suitable manner. In one embodiment, the computer system comprises a stand-alone system. In another embodiment, the computer system is part of a network of computers including a server and a database.

The software required for receiving, processing, and analyzing information may be implemented in a single device or implemented in a plurality of devices. The software may be accessible via a network such that storage and processing of information takes place remotely with respect to users. The system according to various aspects of the present invention and its various elements provide functions and operations to facilitate detection and/or analysis, such as data gathering, processing, analysis, reporting and/or diagnosis. For example, in the present aspect, the computer system executes the computer program, which may receive, store, search, analyze, and report information relating to the human genome or region thereof. The computer program may comprise multiple modules performing various functions or operations, such as a processing module for processing raw data and generating supplemental data and an analysis module for analyzing raw data and supplemental data to generate quantitative assessments of a disease status model and/or diagnosis information.

The procedures performed by the system may comprise any suitable processes to facilitate analysis and/or cancer diagnosis. In one embodiment, the system is configured to establish a disease status model and/or determine disease status in a patient. Determining or identifying disease status may include generating any useful information regarding the condition of the patient relative to the disease, such as performing a diagnosis, providing information helpful to a diagnosis, assessing the stage or progress of a disease, identifying a condition that may indicate a susceptibility to the disease, identify whether further tests may be recommended, predicting and/or assessing the efficacy of one or more treatment programs, or otherwise assessing the disease status, likelihood of disease, or other health aspect of the patient.

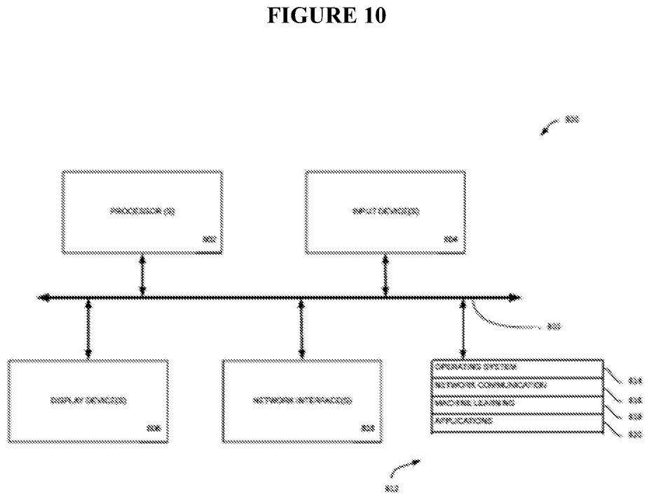

FIG. 10 illustrates an example computer 800 that may be used in predicting the cancer status of a subject. For example, the computer 800 may include a machine learning system that trains a machine learning model to predicting the cancer status of a subject as described above or a portion or combination thereof in some embodiments. The computer 800 may be any electronic device that runs software applications derived from compiled instructions, including without limitation personal computers, servers, smart phones, media players, electronic tablets, game consoles, email devices, etc. In some implementations, the computer 800 may include one or more processors 802, one or more input devices 804, one or more display devices 806, one or more network interfaces 808, and one or more computer-readable mediums 812. Each of these components may be coupled by bus 810, and in some embodiments, these components may be distributed among multiple physical locations and coupled by a network.

Display device 806 may be any known display technology, including but not limited to display devices using Liquid Crystal Display (LCD) or Light Emitting Diode (LED) technology. Processor(s) 802 may use any known processor technology, including but not limited to graphics processors and multi-core processors. Input device 804 may be any known input device technology, including but not limited to a keyboard (including a virtual keyboard), mouse, track ball, camera, and touch-sensitive pad or display. Bus 810 may be any known internal or external bus technology, including but not limited to ISA, EISA, PCI, PCI Express, USB, Serial ATA or FireWire. Computer-readable medium 812 may be any non-transitory medium that participates in providing instructions to processor(s) 804 for execution, including without limitation, non-volatile storage media (e.g., optical disks, magnetic disks, flash drives, etc.), or volatile media (e.g., SDRAM, ROM, etc.).

Computer-readable medium 812 may include various instructions 814 for implementing an operating system (e.g., Mac OS®, Windows®, Linux). The operating system may be multi-user, multiprocessing, multitasking, multithreading, real-time, and the like. The operating system may perform basic tasks, including but not limited to: recognizing input from input device 804; sending output to display device 806; keeping track of files and directories on computer-readable medium 812; controlling peripheral devices (e.g., disk drives, printers, etc.) which can be controlled directly or through an I/O controller; and managing traffic on bus 810. Network communications instructions 816 may establish and maintain network connections (e.g., software for implementing communication protocols, such as TCP/IP, HTTP, Ethernet, telephony, etc.).

Machine learning instructions 818 may include instructions that enable computer 800 to function as a machine learning system and/or to training machine learning models to generate DMS values as described herein. Application(s) 820 may be an application that uses or implements the processes described herein and/or other processes. The processes may also be implemented in operating system 814. For example, application 820 and/or operating system may create tasks in applications as described herein.

The described features may be implemented in one or more computer programs that may be executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program may be written in any form of programming language (e.g., Objective-C, Java), including compiled or interpreted languages, and it may be deployed in any form, including as a stand-alone program or as a module, component, subroutine, or other unit suitable for use in a computing environment.

Suitable processors for the execution of a program of instructions may include, by way of example, both general and special purpose microprocessors, and the sole processor or one of multiple processors or cores, of any kind of computer. Generally, a processor may receive instructions and data from a read-only memory or a random access memory or both. The essential elements of a computer may include a processor for executing instructions and one or more memories for storing instructions and data. Generally, a computer may also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data may include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory may be supplemented by, or incorporated in, ASICs (application-specific integrated circuits).

To provide for interaction with a user, the features may be implemented on a computer having a display device such as an LED or LCD monitor for displaying information to the user and a keyboard and a pointing device such as a mouse or a trackball by which the user can provide input to the computer.

The features may be implemented in a computer system that includes a back-end component, such as a data server, or that includes a middleware component, such as an application server or an Internet server, or that includes a front-end component, such as a client computer having a graphical user interface or an Internet browser, or any combination thereof. The components of the system may be connected by any form or medium of digital data communication such as a communication network. Examples of communication networks include, e.g., a telephone network, a LAN, a WAN, and the computers and networks forming the Internet.

The computer system may include clients and servers. A client and server may generally be remote from each other and may typically interact through a network. The relationship of client and server may arise by virtue of computer programs running on the respective computers and having a client-server relationship to each other.

One or more features or steps of the disclosed embodiments may be implemented using an API. An API may define one or more parameters that are passed between a calling application and other software code (e.g., an operating system, library routine, function) that provides a service, that provides data, or that performs an operation or a computation.

The API may be implemented as one or more calls in program code that send or receive one or more parameters through a parameter list or other structure based on a call convention defined in an API specification document. A parameter may be a constant, a key, a data structure, an object, an object class, a variable, a data type, a pointer, an array, a list, or another call. API calls and parameters may be implemented in any programming language. The programming language may define the vocabulary and calling convention that a programmer will employ to access functions supporting the API.

In some implementations, an API call may report to an application the capabilities of a device running the application, such as input capability, output capability, processing capability, power capability, communications capability, etc.

While various embodiments have been described above, it should be understood that they have been presented by way of example and not limitation. It will be apparent to persons skilled in the relevant art(s) that various changes in form and detail can be made therein without departing from the spirit and scope. In fact, after reading the above description, it will be apparent to one skilled in the relevant art(s) how to implement alternative embodiments. For example, other steps may be provided, or steps may be eliminated, from the described flows, and other components may be added to, or removed from, the described systems. Accordingly, other implementations are within the scope of the following claims.

In addition, it should be understood that any figures which highlight the functionality and advantages are presented for example purposes only. The disclosed methodology and system are each sufficiently flexible and configurable such that they may be utilized in ways other than that shown.

Although the term “at least one” may often be used in the specification, claims and drawings, the terms “a”, “an”, “the”, “said”, etc. also signify “at least one” or “the at least one” in the specification, claims and drawings.

Finally, it is the applicant's intent that only claims that include the express language “means for” or “step for” be interpreted under 35 U.S.C. 112(f). Claims that do not expressly include the phrase “means for”or “step for”are not to be interpreted under 35 U.S.C. 112(f).

The presently described methods and systems are useful for detecting, predicting, treating and/or monitoring cancer status in a subject. Any appropriate subject, such as a mammal can be assessed, monitored, and/or treated as described herein. Examples of some mammals that can be assessed, monitored, and/or treated as described herein include, without limitation, humans, primates such as monkeys, dogs, cats, horses, cows, pigs, sheep, mice, and rats. For example, a human having, or suspected of having, cancer can be assessed using a method described herein and, optionally, can be treated with one or more cancer treatments as described herein.

The following example is provided to further illustrate the embodiments of the present invention but are not intended to limit the scope of the invention. While this is typical of the methods that might be used, other procedures, methodologies, or techniques known to those skilled in the art may alternatively be used.

### EXAMPLES

### Example 1

### Incorporating Clinical Risk Into Biomarker-Based Assessment Used as a Prescreen Prior to LDCT Lung Cancer Screening

Personalized risk assessment could improve the net benefit of LDCT screening because the probability of screening benefit varies in the population with smoking history. The risk of lung cancer can be estimated from clinical factors, including age and smoking history. However, blood-based biomarkers have shown promise to substantially improve risk estimation beyond clinical risk. Blood-based biomarker assessments that identify genomic signatures of lung cancer, if used as a prescreen, could improve the efficiency of LDCT screening.

Among those eligible, such an assessment could distinguish between individuals more and less likely to have lung cancer found by LDCT. In clinical use, genomic signatures are typically interpreted using a cutpoint, above which results are positive, and below negative. But relying solely on a genomic signature ignores underlying clinical risk factors differences such as age and smoking history. Here it is shown that the integration of individual-level clinical risk with genomic signatures of lung cancer, improves the identification of people who are most likely to have lung cancer found by screening.

### Study Participants

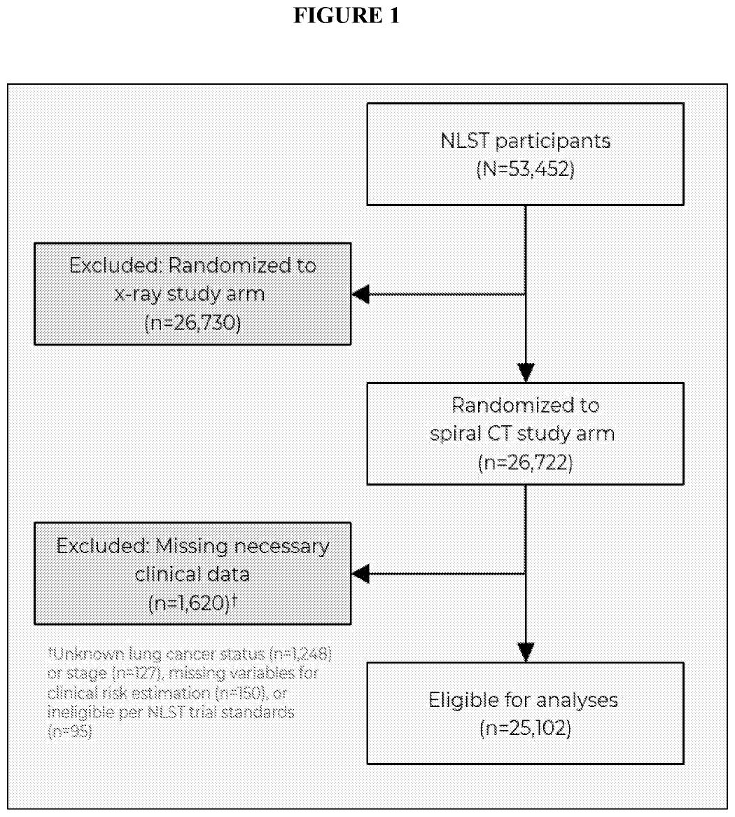

The data from the current study includes participants of the National Lung Screening Trial (NLST). In total, there were 53,452 NLST participants enrolled in the NLST. (See FIG. 1, top panel). 26,730 participants were randomized to the x-ray study arm, while 26,722 participants were randomized to the spiral CT study arm. The former group (the x-ray study arm) was excluded from the current analysis. Additionally, 1,620 participants from the spiral CT study arm were excluded from the current analysis because they missed necessary clinical data. In total, 25,102 participants were eligible for the current analysis. (See FIG. 1, bottom panel).

### Determining Clinical Risk

For the eligible participants, the Bach lung cancer incidence model was used to estimate 1-year lung cancer risk for each participant, as described in Bach, P. B., et al. J NATL CANCER INST. 95(6):470-8 (2003), which is herein incorporated with respect to its description of the Bach lung cancer incidence model. The 25th percentile of clinical risk was chosen as the cutpoint separating low from high clinical risk. The 1-year observed lung cancer diagnosis was predicted in logistic regression models: one with the genomic signature score alone, one with the clinical risk category alone, and one incorporating both genomic and clinical risk category. The models were compared in terms of their specificity at sensitivity of 80% and the number of CT scans needed to detect one lung cancer at an overall prevalence of 1%. Wilson Score confidence intervals were estimated.

### Determining Genomic Risk

A genomic signature score was simulated for each participant. Scores were drawn from distributions of cohorts assessed using DELFI technology, stratified by cancer status and disease stage. DELFI technology evaluates the fragmentation profiles of cell-free DNA present in the blood and uses supervised machine-learning to detect signals of cancer.

Statistical Analyses Clinical risk was considered as a continuous predictor of one-year observed lung cancer in two additional logistic regression models: one with the clinical risk alone, and one incorporating both genomic and clinical risk. Predicted probabilities for the outcome of lung cancer were ascertained from the logistic regression models with continuous predictors. To stratify the predicted probabilities from the multivariable models, a threshold was calculated at 80% sensitivity. The models were compared in terms of their specificity at sensitivity of 80% and the number of CT scans needed to detect one lung cancer at an overall prevalence of 1%. 95% confidence intervals (“CI”) were calculated using bootstrap sampling.

### Results

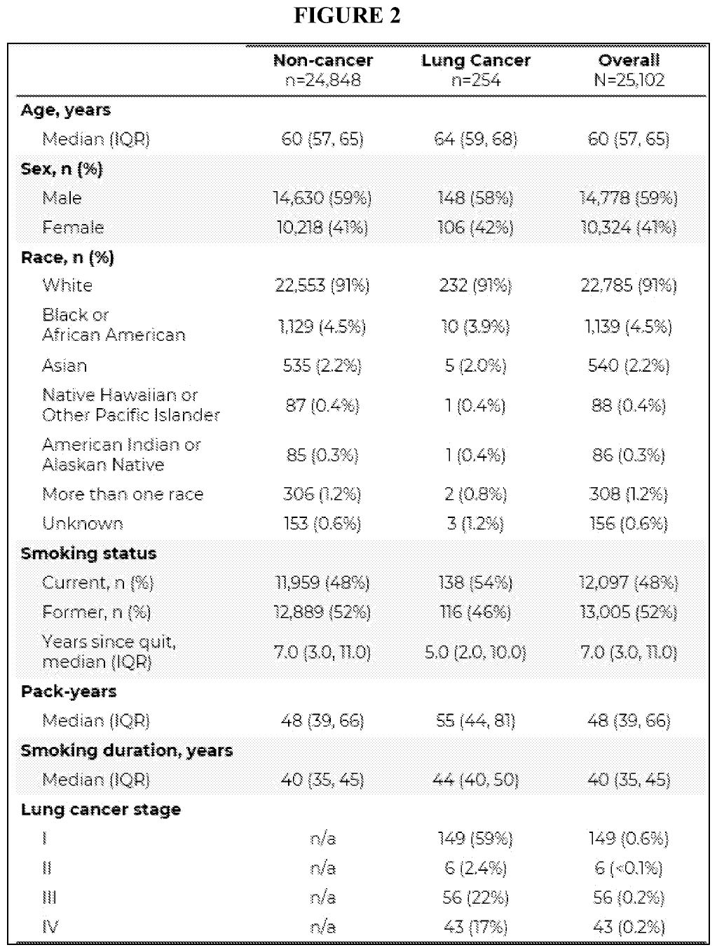

The analysis included 25,102 subjects, 254 (1.0%) of whom were diagnosed with lung cancer within one year. (See FIG. 2). Median (interquartile range) clinical risk was 0.39% (0.23 to 0.63). Thus, the cutpoint separating low and high clinical risk was 0.23%. Median risk of lung cancer was 0.15% (low-risk group) and 0.49% (high-risk group).

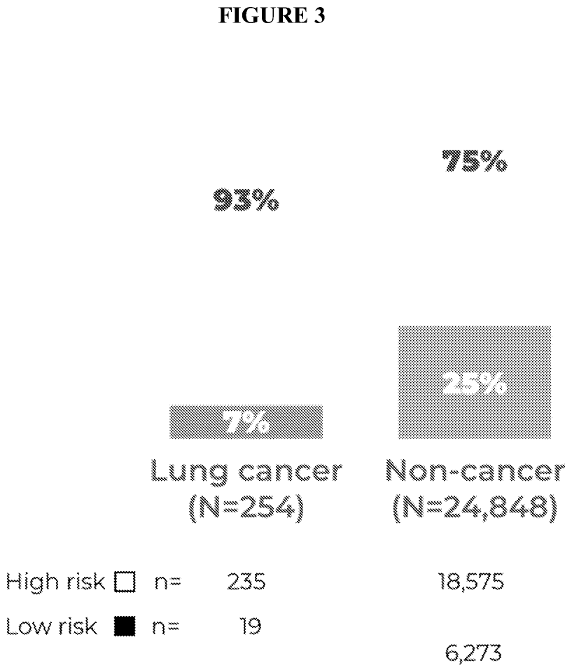

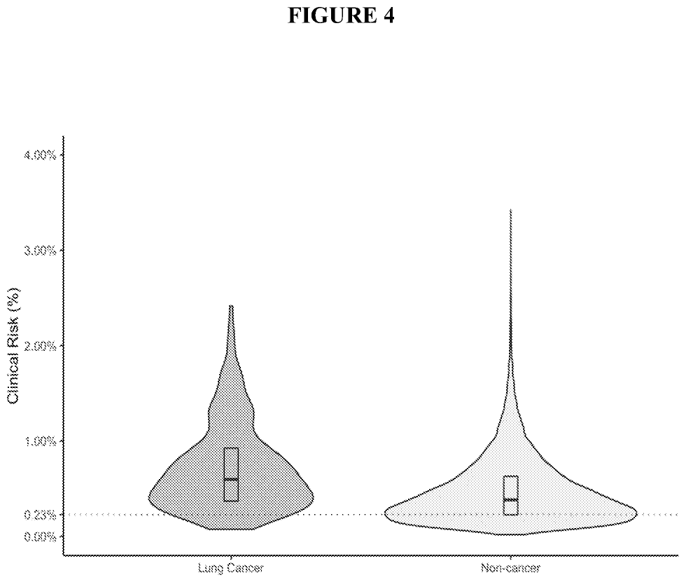

Models incorporating simulated genomic risk, clinical risk, and both combined were all significantly associated with lung cancer diagnosis. (p<0.0001). For example, FIG. 3 illustrates binary clinical risk by cancer status, whereas FIG. 4 illustrates the distribution of clinical risk by cancer status. The latter (FIG. 4), illustrates that median clinical risk was 0.60% (0.37 to 0.93) in the lung cancer group and 0.38% (0.23 to 0.63) in the non-cancer group. The line inside the rectangular box in FIG. 4 represents the median (the line) and the IQR (the rectangular box), respectively.

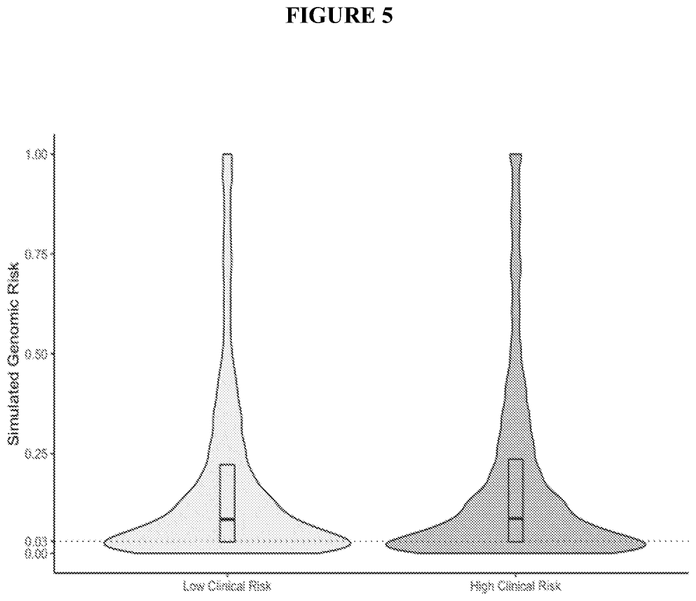

FIG. 5 illustrates the distribution of simulated genomic risk by clinical risk status. In both the low and high clinical risk groups, simulated genomic risk scores ranged from 0 to 1. Similarly here, the line inside the rectangular box in FIG. 5 represents the median (the line) and the IQR (the rectangular box), respectively.

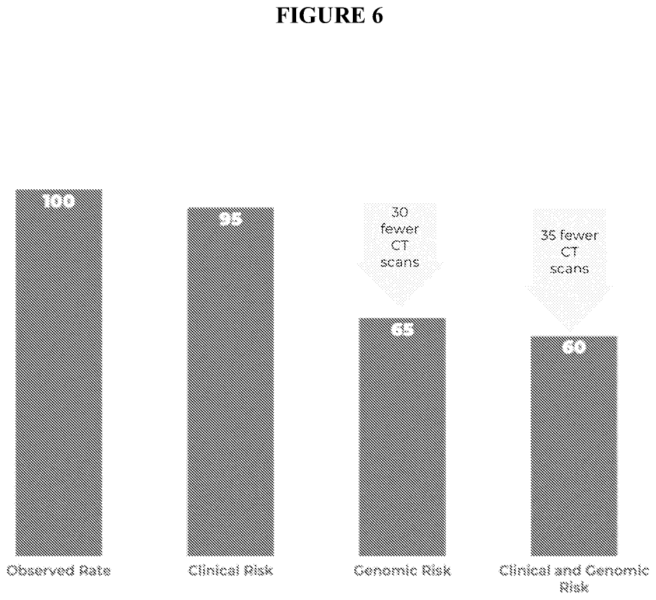

FIG. 6 illustrates the number of CT scans needed to detect one lung cancer by type of risk estimation. The observed rate of CT scans needed to detect one lung cancer was calculated from the prevalence of lung cancer in the NLST CT arm. Compared with categorical clinical risk alone, using genomic risk alone reduced the number of CT scans needed by 32%, from 95 to 65. Combining genomic and categorical clinical risk reduced the number by 37%, from 95 to 60.

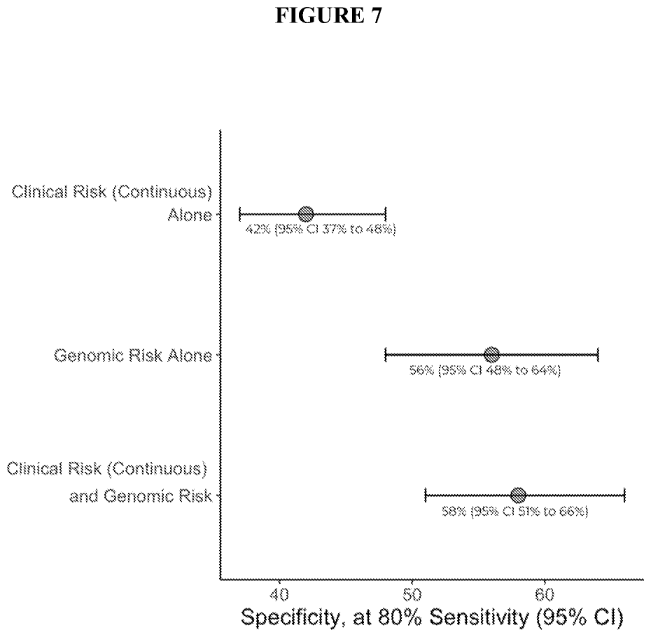

FIG. 7 illustrates that incorporating clinical risk into genomic risk increased specificity from 56% (95% CI 0.55 to 0.57) to 59% (95% CI 0.58 to 0.60) at 80% sensitivity and decreased the number of CT scans needed to detect a single lung cancer from 65 (genomic risk alone) to 60, a 7% reduction in the number needed to screen with LDCT. For reference, without any pre-screen assessment, the number needed to screen to detect one lung cancer was approximately 100.

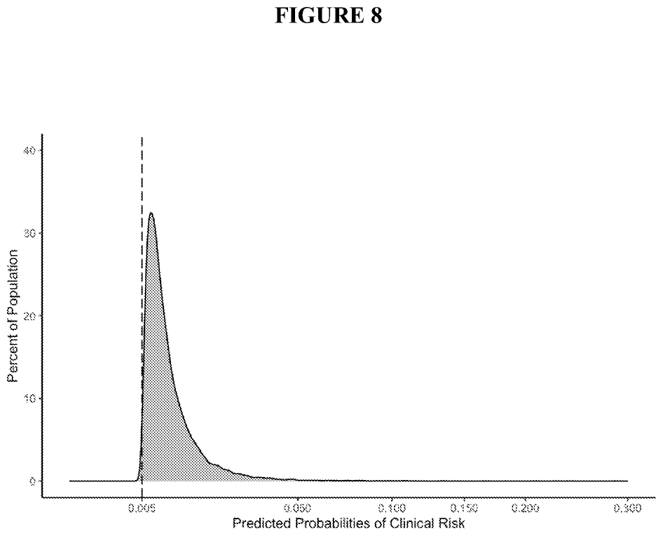

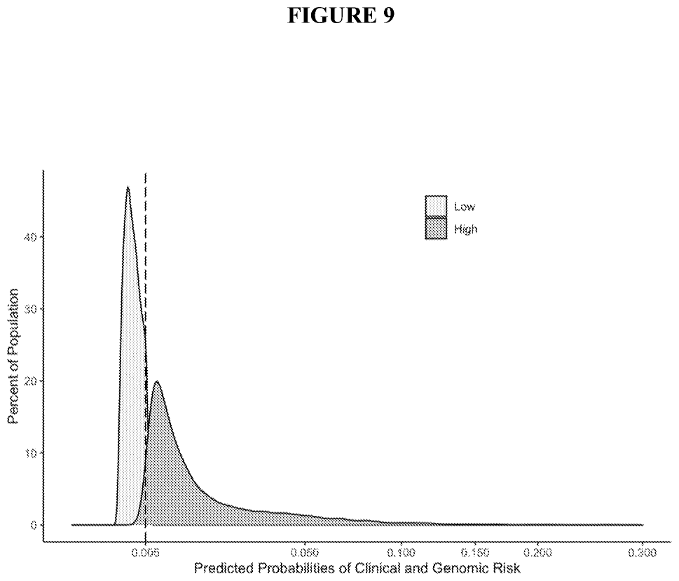

FIG. 8 illustrates the predicted probabilities of lung cancer diagnosis, using clinical risk, while FIG. 9 illustrates predicted probabilities of lung cancer diagnosis, using clinical and genomic risk. The threshold at 80% sensitivity separating low and high predicted probability of lung cancer diagnosis with combined clinical (continuous) and genomic risk was set at 0.005 (dotted line). Incorporating clinical risk into genomic risk allowed for further discrimination between those who fall below and above the threshold.

1. A method of predicting the cancer status of a subject comprising:
a) determining a clinical risk score for the subject;
b) determining a genomic risk score the subject;
c) combining the clinical risk score with the genomic risk score, thereby predicting the cancer status of the subject.

2. The method of claim 1, wherein the clinical score comprises the age, sex and/or race of the subject.

3. The method of claim 1, wherein the genomic risk score comprises cell free DNA (cfDNA) fragment size density data from the subject.

4. The method of claim 3, wherein determining the cfDNA fragment size density data for the subject comprises:
a) processing a sample from the subject comprising cfDNA fragments into libraries;
b) subjecting the libraries to low-coverage whole genome sequencing to obtain sequenced fragments;
c) mapping the sequenced fragments to a genome to obtain windows of mapped sequences;
d) analyzing the windows of mapped sequences to determine cfDNA fragment lengths; and
e) generating the cfDNA fragment size density data.

5. The method of claim 4, wherein the cfDNA fragment size density data comprises a cfDNA fragment size density curve, wherein the cfDNA fragment size density curve from the subject is compared to a cfDNA fragment size density curve from a known healthy subject and/or a known cancer patient.

6-9. (canceled)

10. The method of claim 1, wherein the sensitivity of cancer prediction is at least about 50%, 60%, 70%, 80%, 90% or more.

11-12. (canceled)

13. The method of claim 1, wherein the cancer is lung cancer.

14. The method of claim 13, wherein the clinical risk score for the subject is determined from data comprising the age, sex, race, smoking status, number of pack years, and smoking duration of the subject.

15. The method of claim 14, wherein the clinical risk score for the subject is determined from data comprising the Bach lung cancer incidence model.

16. The method of claim 1, wherein the clinical risk score and the genomic risk score result in a combined score that increases as the subject's risk for cancer increases or decreases as the subject's risk for cancer decreases.

17. (canceled)

18. The method of claim 4, wherein the cfDNA fragment size density data is calculated for a subgenomic interval.

19. The method of claim 1, wherein a subject predicted to have cancer is administered a cancer treatment.

20. The method of claim 3, wherein the cfDNA is obtained from a blood sample from the subject.

21. The method of claim 4, wherein the mapped sequences comprise tens to thousands of windows.

22. The method of claim 4, wherein the windows are non-overlapping windows.

23. The method of claim 4, wherein the windows each comprise about 5 million base pairs.

24. The method of claim 4, wherein a cfDNA fragmentation profile is determined within each window.

25. The method of claim 24, wherein the cfDNA fragmentation profile comprises a fragment size of greatest frequency.

26. The method of claim 24, wherein the cfDNA fragmentation profile comprises a fragment size distribution having fragment sizes of varying frequency.

27. The method of claim 24, wherein the cfDNA fragmentation profile comprises a ratio of small cfDNA fragments to large cfDNA fragments in said windows of mapped sequences.

28. The method of claim 24, wherein the cfDNA fragmentation profile comprises the sequence coverage of small cfDNA fragments, large cfDNA fragments or a combination thereof in windows across the genome.

29-30. (canceled)

31. The method of claim 24, wherein the cfDNA fragmentation profile is over the whole genome or a subgenomic interval.

32. (canceled)

33. The method of claim 19, wherein the cancer treatment is selected from a surgical intervention, adjuvant chemotherapy, neoadjuvant chemotherapy, radiation therapy, hormone therapy, cytotoxic therapy, immunotherapy, adoptive T cell therapy, targeted therapy, a kinase inhibitor, an antibody, signal transduction inhibitors, bispecific antibodies or antibody fragments, monoclonal antibodies, immune checkpoint inhibitors, surgery, or a combination thereof.
